(19) United States
(12) Patent Application Publication
(10) Pub. No.: US 2016/0015534 A1
(43) Pub. Date: Jan. 21, 2016

(54) STENT

(71) Applicant: TERUMO KABUSHIKI KAISHA, Tokyo (JP)

(72) Inventors: Taisei KAWAKITA, Fujinomiya-shi (JP); Hideaki YAMASHITA, Fuji-shi (JP)

(73) Assignee: TERUMO KABUSHIKI KAISHA, Tokyo (JP)

(21) Appl. No.: 14/867,623

(22) Filed: Sep. 28, 2015

Related U.S. Application Data
(63) Continuation of application No. PCT/JP2013/059852, filed on Apr. 1, 2013.

(51) Int. Cl. A61F 2/82

(52) U.S. Cl. CPC A61F 2/82; A61F 2002/825; A61F 2210/0076

(57) ABSTRACT

A stent which improves safety by allowing a living body to be less affected even if a drug is detached. A stent has a strut that is formed in a linear shape, and that defines a cylindrical shape having an opening, a side surface coating member which coats at least a part of at least one of the side surfaces of the strut, the side surface coating member including a drug and a drug loading member which is a polymeric material for loading the drug, and an outer surface coating member which coats at least a part of the outer surface of the strut, the outer surface coating member including the drug without including the drug loading member.

### CROSS-REFERENCE TO RELATED APPLICATION

This application is a continuation of International Application No. PCT/JP2013/059852 filed on Apr. 1, 2013, the entire content of which is incorporated herein by reference.

### TECHNICAL FIELD

The disclosure herein relates to a stent which is caused to indwell in a stenosis, an occluded area, or the like, occurring in a living body lumen so as to maintain a patency of a lumen.

### BACKGROUND DISCUSSION

In recent years, in order to treat myocardinal infarction or angina pectoris, a method has been used in which a stent is caused to indwell in a lesion area (stenosis) of a coronary artery so as to secure a space inside an artery. In some cases, in order to treat a stenosis occurring in other living body lumens such as other blood vessels, biliary ducts, bronchial tubes, esophagi, and urethrae, a similar method has been used. The type of stent used is generally either a balloon-expandable stent or a self-expandable stent, depending on the function and method for indwelling the stent.

In a case of the balloon-expandable stent, the stent itself has no expandable function. After being inserted into a target portion, the stent is expanded and plastically deformed by a balloon. In this manner, the stent is fixedly and closely attached to the inside of the lumen. In contrast, in a case of the self-expandable stent, the stent itself has the expandable function. The stent is accommodated in a catheter in a state where the stent has a decreased diameter prior to use. After reaching a target portion, the stent is expanded by releasing the state having the decreased diameter. In this manner, the stent is fixedly and closely attached to the inside of the lumen. For example, Japanese Patent No. JP-A-H11-313893 discloses a method in which the self-expandable stent having the decreased diameter is accommodated inside an outer sheath, and in the target portion, the outer sheath is pulled toward an operator so as to push out the stent from the outer sheath and to expand the stent.

However, even when the stent expands the stenosis by indwelling the stent in the target portion in this way, in some cases, a restenosis appears in the stent indwelling portion. The main cause of the restenosis is the growth of a vascular intima which is a healing response of a vascular wall. Therefore, recently, drug eluting-type stents, so called drug eluting stents (DES) have been developed which prevent the restenosis by coating an outer surface of the stent with a drug capable of suppressing the growth of the vascular intima and by eluting the drug in the stent indwelling area. For example, International PCT Publication No. 2010/032643 discloses a configuration in which a drug coated layer configured to include a mixture of a drug and a polymeric material for loading the drug is disposed on an outer surface (surface in contact with the living body lumen) and on a side surface (surface adjacent to the outer surface) of a strut which is a wire member configuring the stent.

However, if the drug coated layer is disposed on the outer surface of the self-expandable stent, when the stent is pushed out from the outer sheath, by pulling the outer sheath accommodating the self-expandable stent for delivery to the proximal side of the outer sheath, there is a possibility that the drug coated layer with which the outer surface of the stent is coated may be detached therefrom due to friction with the outer sheath. Since the drug coated layer contains a polymeric material which is foreign to a living body, it is undesirable that the drug coated layer is detached and falls into the living body.

### SUMMARY

The disclosure herein is made in order to solve the above-described problem, and to provide a stent which can improve safety by allowing a living body to be less affected even if a drug is detached.

A stent according to the disclosure has a strut that is formed in a linear shape, and which defines a cylindrical shape having an opening, a side surface coating member which coats at least a part of at least one of the side surfaces of the strut, the side surfaces adjoining an outer surface of the strut and the side surface coating member including a drug and a drug loading member which is a polymeric material for loading the drug, and an outer surface coating member which coats the outer surface of the strut and which includes the drug without including the drug loading member.

According to the stent configured as described above, when the stent is pushed out from the sheath for delivering the stent, the outer surface coating member, which is likely to be detached and fall down by sliding on an inner surface of the sheath, does not include the drug loading member containing the polymeric material. Accordingly, safety is improved by allowing a living body to be less affected even if the outer surface coating member is detached. Further, when the stent is pushed out from the sheath for delivering the stent, the side surface coating member which does not slide on the inner surface of the sheath includes the drug loading member. Accordingly, it is possible to suppress the occurrence of restenosis or late stent thrombosis by allowing the drug to be gradually eluted and by suppressing cell growth in accordance with the progress of cell formation after the stent indwells.

When the inner surface on the side opposite to the outer surface of the strut is not coated with the drug, the overall stent is quickly coated with cells, and the stent is not exposed inside a living body lumen. Accordingly, it is possible to suppress the occurrence of restenosis or late stent thrombosis.

When the drug loading member is a biodegradable polymer, the drug loading member is gradually biodegraded, and the drug is gradually eluted, thereby suppressing the occurrence of restenosis or late stent thrombosis in the stent indwelling portion.

When the outer surface coating member is configured to include only the drug, the drug causes an excellent and instantaneous effect.

When the drug included in the outer surface coating member is different from the drug included in the side surface coating member, the drug acts in various ways depending on its location on the stent. Accordingly, it is possible to effectively suppress the occurrence of restenosis or late stent thrombosis.

When the drug included in the outer surface coating member is an anticancer drug and the drug included in the side surface coating member is an immunosuppressive drug, the anticancer drug strongly acting on the living body is caused to act on the outer surface coating member which shows an instantaneous effect of the drug, and the immunosuppressive drug is caused to gently act on the living body on the side surface coating member in which the drug is gradually eluted. Accordingly, it is possible to more effectively suppress the occurrence of restenosis or late stent thrombosis.

### BRIEF DESCRIPTION OF THE DRAWINGS

FIG. 1 is a plan view of a stent according to an exemplary embodiment of the disclosure when the stent is expanded.

FIG. 2 is a plan view of the stent according to the exemplary embodiment of the disclosure when the stent is contracted and a diameter of the stent is decreased.

FIG. 3 is an enlarged perspective view illustrating a part of the stent.

FIG. 4 is a cross-sectional view taken along line 4-4 in FIG. 3.

FIG. 5 is a plan view illustrating a stent delivery system for causing the stent to indwell in a living body.

FIG. 6 is a cross-sectional view illustrating a distal portion of the stent delivery system.

FIG. 7 is a cross-sectional view illustrating a case when the stent delivery system causes the stent to indwell in the living body.

### DETAILED DESCRIPTION

Hereinafter, an exemplary embodiment according to the disclosure will be described with reference to the drawings. In some cases, dimensional proportions in the drawings may be exaggerated and different from actual proportions for convenience of description.

A stent 10 according to the exemplary embodiment is used in order to treat stenosis or an occluded area occurring in blood vessels, biliary ducts, bronchial tubes, esophagi, urethrae, or other living body lumens. In this description, a side inserted into a lumen is referred to as a “distal end” or a “distal side”, and an operating hand side is referred to as a “proximal end” or a “proximal side”.

The stent 10 is a so-called self-expandable stent which expands using self-elastic force, and includes a strut 20 which extends in a thin linear shape and a coating member 30 with which the strut 20 is coated.

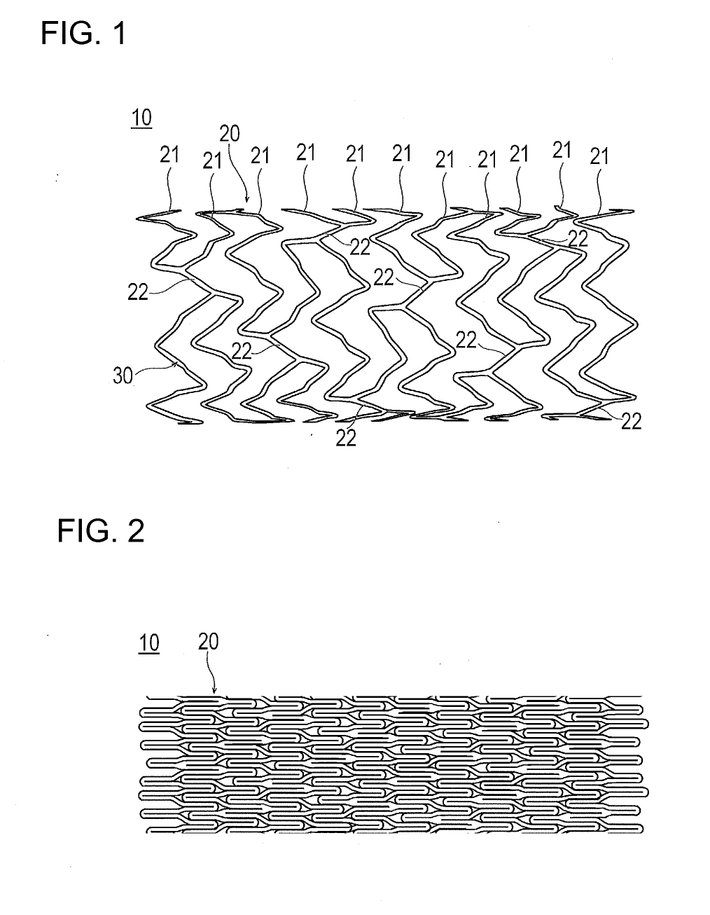

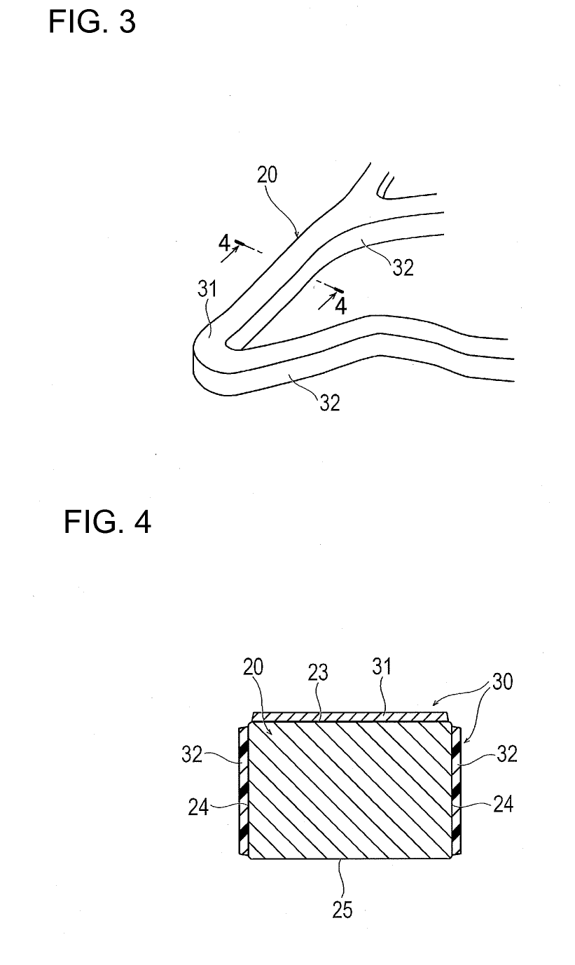

As illustrated in FIGS. 1 to 3, the strut 20 is formed in a cylindrical shape as a whole in such a way that multiple annular portions 21, in which wire members are repeatedly folded and formed in an annular shape, are arrayed in a central axis direction thereof. The adjacent annular portions 21 are integrated with each other by multiple sharing portions 22 shared by the mutual annular portions 21. The number of annular portions 21 is not particularly limited.

As illustrated in FIG. 4, a cross-sectional shape of the strut 20, orthogonal to an extending direction of the strut 20, is rectangular.

The coating member 30 with which the strut 20 is coated includes an outer surface coating member 31 and a side surface coating member 32 on each side.

Both side surfaces 24 of the strut 20, adjoining an outer surface 23 of the strut 20 on a side where the strut 20 is in contact with a living body lumen, are coated with the side surface coating member 32. The side surface coating member 32 includes a drug and a drug loading member which is a polymeric material for loading the drug. The entirety of both side surfaces 24 of the strut 20 may be coated with the side surface coating member 32, only a part of both side surfaces 24 may be coated with the side surface coating member 32, or only one entire side surface 24, or a portion thereof, may be coated with the side surface coating member 32.

The outer surface 23 of the strut 20 is coated with the outer surface coating member 31. The outer surface coating member 31 includes the drug, but does not include the polymeric material serving as the drug loading member included in the side surface coating member 32. All of the outer surface 23 of the strut 20 may be coated with the outer surface coating member 31, or only a part of the outer surface 23 may be coated with the outer surface coating member 31.

An inner surface 25 of the strut 20, on a side opposite to the outer surface 23 of the strut 20, is not coated with the drug, and the strut 20 is exposed therefrom.

Although dimensions of the stent 10 vary depending on its indwelling target portion, in general, the outer diameter when restored or expanded (when the diameter is not decreased) is 1.5 mm to 30 mm, and preferably 2.0 mm to 20 mm. The thickness is 0.04 mm to 1.0 mm, and preferably 0.06 mm to 0.5 mm. The length is 5 mm to 250 mm, and preferably 10 mm to 200 mm.

The thickness of the outer surface coating member 31 is 1 μm to 300 μm, and preferably 3 μm to 100 μm. The thickness of the side surface coating member 32 is 1 μm to 300 μm, and preferably 3 μm to 100 μm.

The strut 20 is integrally formed of super-elastic metal which exhibits superelasticity either both before the strut 20 is inserted into the living body and after the strut 20 is inserted into the living body, in a substantially cylindrical shape.

As the super-elastic metal, a super-elastic alloy is preferably used. The super-elastic alloy is generally called a shape memory alloy, and shows superelasticity in the environment of at least living body temperature (approximately 37° C.). It is preferable to use a super-elastic metal body such as a Ti—Ni alloy containing Ni of 49 atomic % to 54 atomic %, a Cu—Zn alloy containing Zn of 38.5 weight % to 41.5 weight %, a Cu—Zn—X (X=Be, Si, Sn, Al, or Ga) alloy containing X of 1 weight % to 10 weight %, a Ni—Al alloy containing Al of 36 atomic % to 38 atomic %, and the like. In particular, the above-described Ti-Ni alloy is preferably used. In addition, mechanical properties can be appropriately changed if a portion of the Ti—Ni alloy is changed to a Ti—Ni—X alloy (X=Co, Fe, Mn, Cr, V, Al, Nb, W, B, Au, Pd, or the like) replaced with X of 0.01 weight % to 10.0 weight %, or if a portion of the Ti—Ni alloy is changed to a Ti—Ni—X alloy (X=Cu, Pb, or Zr) replaced with X of 0.01 atomic % to 30.0 atomic %, or alternatively if conditions of a cold working ratio and/or final heat treatment are selected.

Buckling stress (yield stress upon loaded) of the super-elastic alloy to be used is 5 kg/mm2 to 200 kg/mm2 (22° C.), and preferably 8 kg/mm2 to 150 kg/mm2. Restoring stress (yield stress upon being unloaded) is 3 kg/mm2 to 180 kg/mm2 (at 22° C.), and preferably 5 kg/mm2 to 130 kg/mm2. Here, the term of superelasticity means that normal metal substantially restores its original shape without heating after the metal is unloaded, even if the metal is deformed (bent, tensioned, or compressed) up to a region of plastic deformation in an operating temperature.

By way of example, the strut 20 is produced by using a super-elastic metal pipe so as to remove (for example, cutting or dissolving) the portion of the pipe which does not form the strut. In this manner, the strut 20 is integrally formed. The super-elastic metal pipe used in forming the strut 20 can be produced as follows. An ingot of the super-elastic alloy is formed in inert gas or vacuum atmosphere. A large diameter pipe is formed by mechanically polishing the ingot, and subsequently by means of hot pressing and extrusion. Thereafter, a die drawing process and a heat treatment process are sequentially and repeatedly performed so that the diameter of the pipe is decreased to a predetermined thickness and outer diameter. Finally, the surface thereof is chemically or physically polished. Then, the strut 20 using the super-elastic metal pipe can be formed by means of cutting work (for example, mechanical polishing or laser cutting), electrical discharge machining, chemical etching, or the like. Furthermore, a combination thereof may also be used.

Examples of drugs included in the outer surface coating member 31 or the side surface coating member 32 include anticancer drugs, immunosuppressive drugs, antibiotics, anti-rheumatic drugs, anti-thrombotic drugs, HMG-CoA reductase inhibitors, insulin resistance improving drugs, ACE inhibitors, calcium antagonists, anti-hyperlipidemic drugs, integrin inhibitors, anti-allergic drugs, anti-oxidants, GP IIb/IIIa antagonists, retinoids, flavonoids, carotenoids, lipid improving drugs, DNA synthesis inhibitors, tyrosine kinase inhibitors, antiplatelet drugs, anti-inflammatory drugs, biologically-derived materials, interferon, and nitric oxide production-promoting substances.

For example, the anticancer drugs include vincristine, vinblastine, vindesine, irinotecan, pirarubicin, paclitaxel, docetaxel, and methotrexate. For example, the immunosuppressive drugs include sirolimus, sirolimus derivatives such as everolimus, pimecrolimus, ABT-578, AP23573, CCI-779, and the like, tacrolimus, azathioprine, cyclosporine, cyclophosphamide, mycophenolate mofetil, gusperimus, mizoribine, and doxorubicin.

For example, the antibiotics include mitomycin, actinomycin, daunorubicin, idarubicin, pirarubicin, aclarubicin, epirubicin, peplomycin, and zinostatin stimalamer. For example, the anti-rheumatic drugs include methotrexate, sodium thiomalate, penicillamine, or lobenzarit. For example, anti-thrombotic drugs include heparin, aspirin, anti-thrombin, ticlopidine, and hirudin.

For example, the HMG-CoA reductase inhibitors include cerivastatin, cerivastatin sodium, atorvastatin, atorvastatin calcium, rosuvastatin, rosuvastatin calcium, pitavastatin, pitavastatin calcium, fluvastatin, fluvastatin sodium, simvastatin, lovastatin, pravastatin, and pravastatin sodium.

For example, the insulin resistance improving drugs include thiazolidine derivatives such as troglitazone, rosiglitazone, pioglitazone, and the like. For example, the ACE inhibitors include quinapril, perindopril erbumine, trandolapril, cilazapril, temocapril, delapril, enalapril maleate, lisinopril, and captopril. For example, the calcium antagonists include nifedipine, nilvadipine, diltiazem, benidipine, and nisoldipine.

For example, the anti-hyperlipidemic drugs include bezafibrate, fenofibrate, ezetimibe, torcetrapib, pactimibe, K-604, implitapide, and probucol.

For example, the integrin inhibitors include AJM300. For example, the anti-allergic drugs include tranilast. For example, the anti-oxidants include a-tocopherol, catechin, dibutyl hydroxy toluene, and butyl hydroxy anisole. For example, the GP IIb/IIIa antagonists include abciximab. For example, the retinoids include all-trans-retinoic acid. For example, the flavonoids include epigallocatechin, anthocyanins, and proanthocyanidins. For example, the carotenoids include [symbol] -carotene and lycopene.

For example, the lipid improving drugs include eicosapentaenoic acid. For example, the DNA synthesis inhibitors include 5-FU. For example, the tyrosine kinase inhibitors include genistein, tyrphostin, erbstatin, and staurosporine. For example, the antiplatelet drugs include ticlopidine, cilostazol, and clopidogrel. For example, the anti-inflammatory drugs include steroid such as dexamethasone, prednisolone, and the like.

For example, the biologically-derived materials include an epidermal growth factor (EGF), a vascular endothelial growth factor (VEGF), a hepatocyte growth factor (HGF), a platelet derived growth factor (PDGF), and a basic fibroblast growth factor (BFGF). For example, the interferon includes interferon-μ1a. For example, the nitric oxide production-promoting substances include L-arginine.

It is preferable to use paclitaxel, docetaxel, sirolimus, and everolimus in view of the fact that all of these are generally used for stenosis treatment and can be efficiently transferred into cells in a short time. In particular, it is preferable to use sirolimus and paclitaxel.

Drugs included in the outer surface coating member 31 may be the same as drugs included in the side surface coating member 32, or may be different therefrom. As an example, the drugs included in the outer surface coating member 31 can be paclitaxel serving as the anticancer drug, and the drugs included in the side surface coating member 32 can be sirolimus serving as the immunosuppressive drug.

The drug loading member includes a polymeric material, for example, polyolefin, polyisobutylene, ethylene-a-olefin copolymer, acrylic polymer, polyvinyl chloride, polyvinyl methyl ether, polyvinylidene fluoride, polyvinylidene chloride, polyacrylonitrile, polyvinyl ketone, polystyrene, polyvinyl acetate, ethylene-methyl-methacrylate copolymer, acrylonitrile-styrene copolymer, ABS resin, polyoxymethylene, polyester, polyether, polyamide, epoxy resin, polyurethane, rayon-triacetate, cellulose, cellulose acetate, cellulose butyrate, cellophane, cellulose nitrate, propionyl cellulose, cellulose ethers, carboxymethyl cellulose, chitin, polylactic acid, polyglycolic acid, polycaprolactone, lactic acid-glycolic acid copolymer, lactic acid-caprolactone copolymer, polyethylene oxide, polylactic acid-polyethylene oxide copolymer, polyethylene glycol, polypropylene glycol, polyvinyl alcohol, and polyvinylpyrrolidone.

In particular, it is preferable that the drug loading member includes a biodegradable polymer which is degraded inside the living body. After the stent 10 indwells in the living body, when the biodegradable polymer loading the drug is biodegraded, the drug is gradually eluted, thereby preventing restenosis in the stent indwelling area. As the biodegradable polymer, it is preferable to use any one of polylactic acid, polyglycolic acid, polycaprolactone, lactic acid-glycolic acid copolymer, and lactic acid-caprolactone copolymer.

When the strut 20 is coated with the outer surface coating member 31, the outer surface 23 of the strut 20 is coated with a coating solution obtained by dissolving the drug in a solvent. In this manner, the outer surface 23 can be coated by evaporating the solvent and by drying and solidifying the drug.

When the strut 20 is coated with the side surface coating member 32, the side surface 24 of the strut 20 is coated with the coating solution obtained by dissolving the drug and the drug loading member in the solvent. In this manner, the side surface 24 can be coated by evaporating the solvent and by drying and solidifying the drug and the drug loading member.

The solvent is not particularly limited, but it is preferable to use an organic solvent such as methanol, ethanol, dioxane, tetrahydrofuran, dimethylformamide, acetonitrile, dimethyl sulfoxide, acetone, and the like.

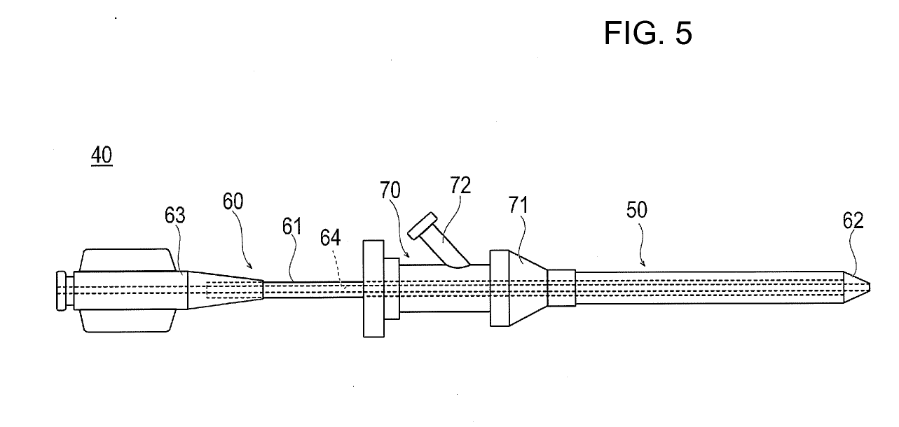

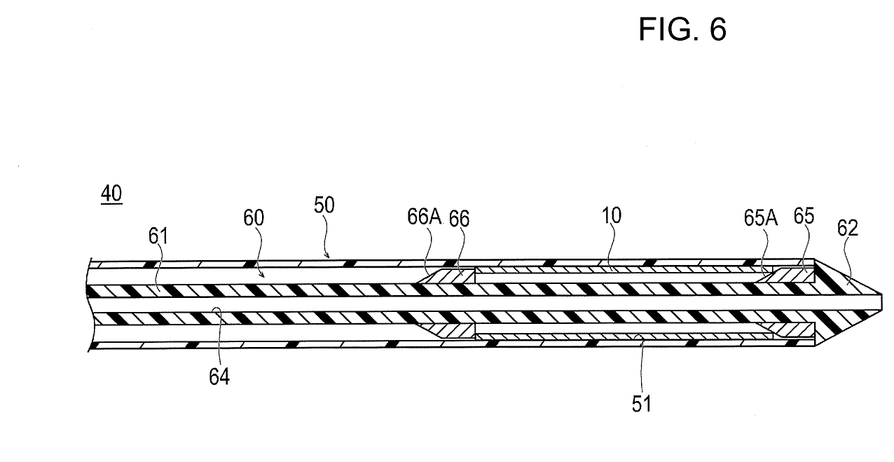

A method will be described in which the stent 10 according to the exemplary embodiment is caused to indwell in the living body lumen. When the stent 10 is caused to indwell, a stent delivery system 40 such as that illustrated in FIGS. 5 and 6 is used.

The stent delivery system 40 includes a tubular sheath 50 and an inner tube 60 which is inserted into the sheath 50 so as to be slidable.

In the sheath 50, the distal end and the proximal end are open, and an accommodation portion 51 which can accommodate the stent 10 is disposed inside on the distal side. When the stent 10 is caused to indwell in the stenosis in the living body lumen, the distal end opening functions as a discharge port of the stent 10. The stent 10 in a state having the decreased diameter is accommodated in the accommodation portion 51, and the outer surface coating member 31 is brought into contact with an inner surface of the accommodation portion 51 of the sheath 50. The stent 10 expands due to its own elastic force after stress is unloaded by the stent 10 being pushed out from the distal end opening, and the stent is restored to a shape which the stent 10 had before being contracted.

In addition, a sheath hub 70 is fixed to the proximal portion of the sheath 50. The sheath hub 70 includes a sheath hub body 71 and a valve (not illustrated) which is accommodated inside the sheath hub body 71 and supports the inner tube 60 so as to be slidable in a liquid-tight manner. In addition, the sheath hub 70 includes a side port 72 which extends obliquely rearward from the vicinity of the center of the sheath hub body 71. In addition, it is preferable that the sheath hub 70 includes an inner tube locking mechanism for restricting the movement of the inner tube 60.

The inner tube 60 includes an inner tube body portion 61 which has a shaft shape, an inner tube distal portion 62 which is disposed in the distal end of the inner tube body portion 61 and protrudes beyond the distal end of the sheath 50, and an inner tube hub 63 which is fixed to the proximal portion of the inner tube body portion 61.

The inner tube distal portion 62 protrudes beyond the distal end of the sheath 50, and is formed in a tapered shape whose diameter gradually decreases toward the distal end. According to this formation, it becomes easy to insert the inner tube distal portion 62 into the stenosis. In addition, since the proximal end of the inner tube distal portion 62 can come into contact with the distal end of the sheath 50, the inner tube distal portion 62 functions as a stopper for preventing the movement in the distal direction of the sheath 50.

A stent holding protrusion portion 65 is disposed on a proximal side of the inner tube distal portion 62 of the inner tube 60. A stent pushing-out protrusion portion 66 is disposed on a proximal side which is spaced by a predetermined distance relative to the stent holding protrusion portion 65. The stent 10 is arranged between the two protrusion portions 65 and 66. It is preferable that the protrusion portions 65 and 66 are annular protrusion portions. Each outer diameter of the protrusion portions 65 and 66 has a size which enables the protrusion portions 65 and 66 to come into contact with the contracted stent 10. Therefore, in the stent 10, the stent holding protrusion portion 65 restricts the movement to the distal side, and the stent extruding protrusion portion 66 restricts the movement to the proximal side. When the sheath 50 is moved to the proximal side in a state where the position of the inner tube 60 is held, the stent pushing-out protrusion portion 66 restricts the movement of the stent 10 to the proximal side. Accordingly, the stent 10 slides on the inner surface of the sheath 50, and is discharged from the sheath 50. Furthermore, it is preferable to configure the proximal side of the stent pushing-out protrusion portion 66 to include a tapered portion 66A whose diameter gradually decreases toward the proximal side. Similarly, it is preferable to configure the proximal side of the stent holding protrusion portion 65 which includes a tapered portion 65A whose diameter gradually decreases toward the proximal side. According to this configuration, it is possible to prevent the protrusion portions 65 and 66 from being locked by the distal end of the sheath 50, when the sheath 50 is moved to the distal side and the inner tube 60 is accommodated inside the sheath 50 again, after the sheath 50 is moved to the proximal side with respect to the inner tube 60 and the stent 10 is discharged from the sheath 50. In addition, the two protrusion portions 65 and 66 may be respectively formed of different members using a material having X-ray contrasting property. In this manner, it is possible to accurately recognize the position of the stent 10 by using X-ray contrast, thereby further facilitating indwelling procedures.

The inner tube 60 penetrates the sheath 50, and protrudes beyond the proximal opening of the sheath 50. The inner tube hub 63 is fixedly attached to the proximal portion of the inner tube 60. In the inner tube 60, a lumen 64 into which a guidewire is inserted is formed so as to extend from the distal end to the proximal end. The lumen 64 may be formed so as to be laterally open from the distal end of the inner tube 60 at an intermediate position of the inner tube 60.

It is preferable to form the sheath 50 using a material which is flexible to some degree. For example, the material includes polyolefin such as polyethylene, polypropylene, polybutene, ethylene-propylene copolymer, ethylene-vinyl acetate copolymer, ionomer, a mixture of two or more of these materials and the like, soft polyvinyl chloride resin, polyamide, polyamide elastomer, polyester, polyester elastomer, polyurethane, fluorine resin such as polytetrafluoroethylene, and the like, silicone rubber, latex rubber, or the like.

The inner tube 60 can employ a material which is similar to that of the sheath 50, or can employ a metal material. Examples of the metal material include stainless steel and a Ni—Ti alloy.

By way of example, the sheath hub 70 and the inner tube hub 63 can employ synthetic resin such as polycarbonate, polyolefin, styrene resin, polyester, and the like, or a metal material such as stainless steel, aluminum, an aluminum alloy, and the like.

When the stent 10 is caused to indwell in the living body lumen (for example, blood vessels) by using the stent delivery system 40, the stent 10 whose diameter decreases toward the central axis is first accommodated in the accommodation portion 51 located on the distal side of the sheath 50. Then, in a state where the stent pushing-out protrusion portion 66 of the inner tube 60 is positioned on the proximal side of the stent 10, the inside of the sheath 50 and the inside of the inner tube 60 are filled with a physiological salt solution.

Next, a sheath introducer is caused to indwell in the blood vessel of a patient by means of a Seldinger technique, for example. In a state where a guidewire is inserted into the guidewire lumen 64, the guidewire and the stent delivery system 40 are inserted into the blood vessel through the inside of the sheath introducer. Subsequently, while the guidewire is caused to advance into the blood vessel, the stent delivery system 40 is moved forward so that the distal portion of the sheath 50 reaches the stenosis.

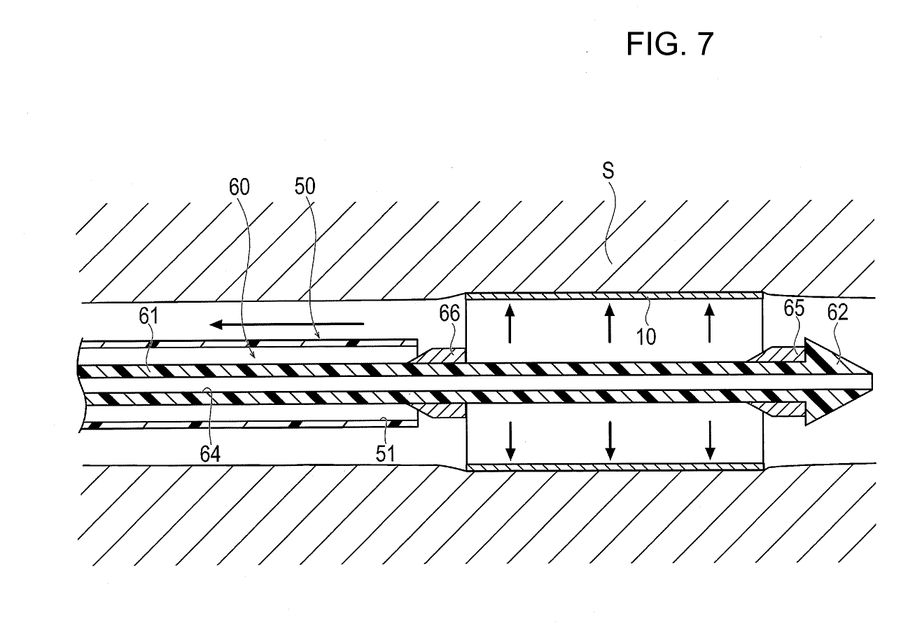

Thereafter, while an operators hand holds down the inner tube 63 so that the stent pushing-out protrusion portion 66 does not move to the proximal side, the sheath hub 70 is pulled and moved to the proximal side. In this manner, the stent 10 is discharged from the distal end opening of the sheath 50 while the sheath 50 is moving in the proximal direction so that the stent 10 is pushed-out by the stent pushing-out protrusion portion 66. In this manner, as illustrated in FIG. 7, the stent 10 expands due to its own elastic force after stress is unloaded, and resumes (is restored to) a shape which the stent 10 had before being contracted. This enables the stent 10 to satisfactorily maintain a state where a stenosis S is spread out by the stent 10.

When the stent 10 is pushed out from the sheath 50, the outer surface 23 of the stent 10 slides on the inner surface of the sheath 50. Accordingly, a part of the outer surface coating member 31 is likely to be detached and fall down. However, since the outer surface coating member 31 does not include the drug loading member having the polymeric material, safety is improved by allowing a living body to be less affected even if the outer surface coating member 31 is detached. Furthermore, since the outer surface coating member 31 does not include the drug loading member, the drug acts more directly on the living body lumen (blood vessel), and the drug causes an excellent and instantaneous effect. In particular, when the outer surface coating member 31 is configured to include only the drug, the drug causes a better and instantaneous effect.

In addition, since the side surface 24 of the stent 10 does not slide on the inner surface of the sheath 50 when the stent 10 is extruded from the sheath 50, not only the side surface coating member 32 but also the drug loading member are less likely to be detached and fall down. Accordingly, it is possible to suppress the growth of vascular endothelial cells in accordance with the progress of cell formation after the stent 10 indwells, by allowing the drug loading member to have a gradually eluted drug in the side surface coating member 32.

Further, since the inner side surface 25 of the stent 10 is not coated with the drug, the overall stent 10 is quickly coated with the vascular endothelial cells, and the stent 10 is not exposed inside the blood vessel. Therefore, it is possible to suppress the occurrence of restenosis or late stent thrombosis.

In addition, when the drug loading member is a biodegradable polymer, the drug loading member is gradually biodegraded, and the drug is gradually eluted. Accordingly, it is possible to suppress the occurrence of restenosis or late stent thrombosis in the indwelling portion of the stent 10.

Also, when the drug included in the outer surface coating member 31 is different from the drug included in the side surface coating member 32, the drug acts in various ways depending on its location on the stent 10. Accordingly, it is possible to effectively suppress the occurrence of restenosis or late stent thrombosis.

In addition, when the drug included in the outer surface coating member 31 is an anticancer drug and the drug included in the side surface coating member 32 is an immunosuppressive drug, the anticancer drug strongly acting on the living body is caused to act on the outer surface coating member 31 which shows an instantaneous effect of the drug, and the immunosuppressive drug is caused to gently act on the living body on the side surface coating member 32 in which the drug is gradually eluted. Accordingly, it is possible to more effectively suppress the occurrence of restenosis or late stent thrombosis.

After the stent 10 indwells in the living body lumen, the guidewire and the stent delivery system 40 are removed from the blood vessel via the sheath introducer, thereby completing the indwelling procedure.

Without being limited to the above-described embodiments, the invention can be modified in various ways within the technical idea of the disclosure herein by those skilled in the art. For example, the inner surface 25 of the strut 20 may be coated with the drug, and the drug may be loaded by the drug loading member. In addition, the outer surface coating member with which the outer surface 23 is coated may include a material other than the polymeric material serving as the drug loading member, in addition to the drug.

In addition, the stent 10 according to the exemplary embodiment is the self-expandable stent. However, a balloon-expandable stent which is expanded by a balloon may be employed. If the disclosure here is applied to the balloon-expandable stent, when the stent is mounted on the balloon and the stent is delivered by the balloon being inserted into the sheath, the outer surface coating member of the stent slides on the inner surface of the sheath. Accordingly, a part of the outer surface coating member is likely to be detached and fall down. However, since the outer surface coating member does not include the drug loading member having the polymeric material, safety is improved by allowing the living body to be less affected.

The detailed description above describes a stent. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents which fall within the scope of the claims are embraced by the claims.

What is claimed is:

1. A stent comprising:
a strut formed in a linear shape and that defines a cylindrical shape having an opening, the strut having opposing side surfaces and an outer surface disposed therebetween;
a side surface coating member which coats at least a part of at least one of the side surfaces of the strut, the side surface coating member including a drug and a drug loading member which is a polymeric material for loading the drug; and
an outer surface coating member which coats at least a part of the outer surface of the strut, the outer surface coating member including the drug without including the drug loading member.

2. The stent according to claim 1,
wherein an inner surface on a side opposite to the outer surface of the strut is not coated with the drug.

3. The stent according to claim 1,
wherein the drug loading member is a biodegradable polymer.

4. The stent according to claim 1,
wherein the outer surface coating member is configured to include only the drug.

5. The stent according to claim 1, wherein the drug included in the outer surface coating member is different from the drug included in the side surface coating member.

6. The stent according to claim 5,
wherein the drug included in the outer surface coating member is an anticancer drug, and the drug included in the side surface coating member is an immunosuppressive drug.

7. The stent according to claim 1, wherein the stent is a self-expandable stent.

8. The stent according to claim 7,
wherein the stent is formed of a super-elastic metal.

9. The stent according to claim 1,
wherein the side surface coating member coats an entirety of both side surfaces of the strut.

10. The stent according to claim 1,
wherein the side surface coating member coats a part of both side surfaces of the strut.

11. The stent according to claim 1,
wherein the outer surface coating member coats an entirety of the outer surface of the strut.

12. The stent according to claim 1,
wherein the drug loading member is a biodegradable polymer.

13. The stent according to claim 1,
wherein a cross-sectional shape of the strut is rectangular.

14. A method of making the stent according to claim 1, comprising: coating the outer surface of the strut with a coating solution obtained by
dissolving the drug in a solvent;
evaporating the solvent and drying and solidifying the drug on the outer surface to form the outer surface coating member on the strut;
coating at least a part of at least one side surface with a coating solution obtained by dissolving the drug and the drug loading member in the solvent; and
evaporating the solvent and drying and solidifying the drug and the drug loading member on the at least one side surface to form the side surface coating member on the strut.
